(19) United States
(12) Patent Application Publication
(10) Pub. No.: US 2020/0377848 A1
(43) Pub. Date: Dec. 3, 2020

(54) RESOURCE RECOVERY METHOD FOR SIMULTANEOUS PRODUCTION OF MICROBIAL INGREDIENT AND TREATED WATER PRODUCTS

(71) Applicant: iCell Sustainable (shanghai) Nutrition Co., Ltd, Shanghai (CN)

(72) Inventors: Weiwei Zhao, Shanghai (CN); Seth Sprague Terry, Englewood, CO (US); Mark Duane Rottmann, Furlong, PA (US); Jianhua Song, Shanghai (CN)

(21) Appl. No.: 16/997,429

(22) Filed: Aug. 19, 2020

(30) Foreign Application Priority Data
May 27, 2020 (CN) 202010461545.8

(51) Int. Cl. C12N 1/20; A23K 10/18; A23K 50/80; C02F 3/02

(52) U.S. Cl. CPC C12N 1/20; A23K 10/18; C02F 2103/32; C02F 3/02; A23K 50/80

(57) ABSTRACT

The present invention discloses a method for producing a nutritional microbial solids product while simultaneously producing clean water for multiples uses. The microbial solids product represents a form of single cell protein (SCP) that finds application most typically in formulated animal feeds, but may also be used in food, fertilizer, or soil amendment products. The treated water product can be used directly or polished further for subsequent industrial or agricultural use, including aquaculture and irrigation. The process described utilizes low-value by-products of industrial production for biochemical conversion into SCP. The by-products most suitable to this approach have high organic content that otherwise makes them difficult to dispose of responsibly via traditional methods such as biological wastewater treatment.

This application claims priority to Chinese Patent Application Ser. No. CN202010461545.8 filed on 27 May 202.

### FIELD OF INVENTION

The present invention relates to a by-product resource recovery method that produces both a valuable microbial biomass ingredient and treated water for multiple uses.

### BACKGROUND

Modern industry produces finished goods through the physical and chemical processing of raw materials that include farm, animal husbandry, fishery, and forestry inputs. This production generates commodities such as for example: grain, meat, aquatic products, beer, and other beverages. Additionally, such production may also yield goods for other industrial fields including chemicals (e.g., monosodium glutamate), fertilizer, soil amendment, energy, and medicine. Underpinning many of these production processes is a fundamental reliance on the water-mediated biochemical conversion of carbon, oxygen, and nutrients into nutritional components that support life at both the macroscopic and microscopic scales.

Humans have long exploited for economic gain this basic biochemical conversion process. However, industrial production based on this process also generates significant wastewater or liquid by-product material of very low value requiring disposal. Focusing for the moment on the food industry, one notes various sources of wastewater that for convenience may be categorized into three broad categories: cleaning, processing, and finishing. The first category includes the removal of unwanted matter and debris from raw materials, generating water-borne compounds comprised for example of silt, leaves, peels, scales, feathers, and hair as well as their component minerals, carbohydrates, lipids, and proteins. The second category includes “hold-up” product in tanks, pipes, and conveyances as well as by-product material with no further use in the main production process. And finally, the third category includes waste produced in final packaging (e.g., spillage during bottling) and disposal of any off-spec product. These categories also see analogies in the other industries noted above, including energy.

In order to mitigate environmental harm, wastewater must be treated. Of interest and great import, biochemical processes also help remove polluting compounds found in wastewater. Towards this end, industries often maintain devoted wastewater treatment facilities (WWTFs) to convert organic compounds into forms that can be discharged safely to the environment. Historically, these treatment facilities served merely as cost-centers built to fulfill regulatory requirements. However, more recently, such treatment works have moved towards realizing the potential of resource recovery and have sought to convert or “upcycle” heretofore unwanted water-borne compounds into commercially valuable products. In so doing, they clearly reduce or otherwise offset treatment and disposal costs.

Particularly where high volumes are involved, biological treatment serves as a standard for removing organic compounds, most often quantified as biochemical oxygen demand (BOD), from wastewater. In such processes, relatively complex organic material can be converted into simpler substances via aerobic microbial metabolism. However, organic by-products produced during the industrial “processing” category noted above often cannot be converted through standard biological treatment. For instance, condensed distillers solubles (CDS), an organic by-product generated from a corn ethanol production, is not amenable to direct microbial treatment for the following reasons: 1) concentrated CDS syrup typically exceeds 500,000 mg_BOD/L (i.e., about 1,000 times that introduced to many WWTFs); 2) concentrated CDS exhibits high viscosity (usually 3,000-4,000 CP), making conveyance and homogeneous mixing an operational challenge; and 3) concentrated CDS can ferment via carryover yeast to produce metabolites recalcitrant to further breakdown.

Industrial biochemical conversion presents numerous other challenging by-products with properties similar to CDS. For instance, palm oil mill effluent (POME) also exhibits very high BOD (generally 30,000-40,000 mg/L); residuals from cane or beet sugar production yields effluent with BOD on the order of 25,000 mg/L or higher; and biodiesel production generates low-quality glycerin with BOD values that regularly overtop 500,000 mg/L. As with CDS, these production by-products have low or no market value and are not amenable to direct treatment to mitigate pollution potential.

To meet the challenge of such industrial by-products, the present invention extends the ability of biological WWTFs to adapt to new inputs. Specifically, under appropriate operation, these facilities may better conform to diverse wastes over a wide range of concentrations. Further, the present invention enables facilities to recover the value inherent in difficult wastes by rendering them available as a substrate for microbial growth. For the purposes of further explanation, these difficult wastes will be referred to as “controlled substrates” for the balance of this document. Generally speaking, this term indicates one or more high-BOD by-products with stable properties during industrial production. With proper pre-treatment as described in the present invention, this non-conventional form of substrate provides the building blocks for microbial metabolism and growth. BOD content of controlled substrates may range from 100,000 mg/L to 1,000,000 mg/L and have viscosity values from 1 CP to 4,000 CP.

This application for patent builds upon a production method for producing single cell protein (SCP) found in U.S. Pat. No. 7,931,806 and China patent CN108192944A. Additionally, the present invention further establishes a production method on this basis that similarly allows for recovery of a treated water product suitable for use as make-up water, diluent, or other applications including agricultural, aquacultural, and industrial. Accordingly, the present invention aligns well with eco-friendly and circular economic approaches by ensuring minimal waste generation as raw materials are either converted into commercial products in their own right or used to enhance production of other goods.

The present invention aims to solve deficiencies found in the prior art by enabling the recovery of valuable resources in the simultaneous production of a microbial ingredient and treated water product. By virtue of the fundamental process involved (i.e., the aqueous biochemical conversion of carbon, oxygen, and nutrients), the two products are derived in the intimate presence of one another. Consequently, the full production process includes the separation of microbial solids from treated water as a supernatant. This treated water product may then be polished further to suit subsequent needs as a diluent, process water, cooling water, growth medium, or irrigation source.

The present invention provides distinct advantages over the prior art for recovering resources from high-BOD or high viscosity wastes that otherwise have little value and may represent a disposal issue. Specifically, the present invention enables upcycling of the resource via microbial processes and thereby delivers valuable microbial solids and clean product water in an economical manner. Further, the present invention solves for cases where certain wastes remain recalcitrant to microbial treatment even when the viscosity of that waste is decreased through dilution. To the point, the present invention provides for pre-treatment of controlled substrates by such techniques as dilution, pulverization, and pH control. Further, such pre-treatment may occur in a high-temperature environment using thermophilic microbes. Relying on thermophiles avoids the costs inherent to high-BOD mesophilic conversion that require expensive cooling systems. Such a high-temperature approach has the further advantage of making products in conformity with food-grade requirements, thereby increasing their commercial value. And finally, in addition to the microbial solids product, the present invention also generates a valuable treated water product that can be polished and tuned for many subsequent uses. As a result, the present invention positions well in industrial settings where zero-waste strategies are valued.

The present invention relates to culturing SCP using controlled substrates. In many ways, this invention builds further upon existing patented technology (see U.S. Pat. No. 7,931,806) for SCP produced during wastewater treatment. However, compared with the patented technology where BOD concentrations in wastewater remain relatively low (i.e., on the order of 500 to 2,500 mg/L), the present invention focuses on far higher BOD substrates. Further, whereas the variable nature of BOD quality and quantity in “standard” wastewater substrate makes producing high-quality food-grade SCP highly challenging, controlled substrates provide for more consistent process control and higher-quality SCP.

### SUMMARY

The present invention relates to the biochemical conversion of high-BOD by-products derived from certain industrial production processes such as found for example in the food, beverage, life sciences, and fuel industries. For reasons discussed, these by-products prove difficult to treat or otherwise dispose of in an environmentally responsible, yet economical manner. The present invention provides for conversion of these by-products into high-value SCP and functional protein for feed and food despite properties of high BOD, high viscosity, low water activity, or other detrimental features such as low pH. Examples for such by-products include concentrated syrup generated from ethanol fermentation (i.e., CDS), wastewater generated from palm oil production (i.e., POME), glycerin generated from biodiesel production, and other similar materials.

The present invention relates to liquid culture mediums composed of at least one controlled substrate used for microbial growth. As discussed, these controlled substrates may be discarded by-products from industrial production. The present invention has the advantage of realizing value from these discarded by-products in an economical and operationally robust manner. Along these lines, application of this technique may be used to downsize or decrease reliance upon expensive standard water purification equipment such as clarifiers or membrane separation system. Additionally, the present invention offers the advantage of producing a valuable treated water product that may find multiple uses. For example, treated wastewater may be recycled back to the main industrial process for use in dilution, cooling, or washing.

### BRIEF DESCRIPTION OF THE DRAWINGS

FIG. 1 shows a diagram of microbial biomass production method.

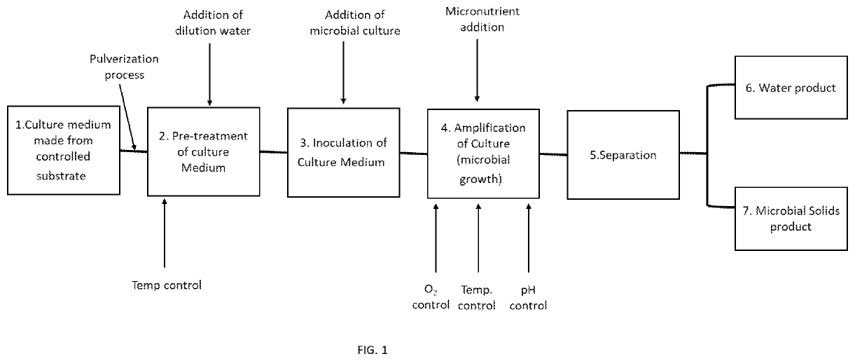

For achieving the purpose of present invention, a microbial biomass production method is described in the FIGURE above comprising:
a culture medium made from controlled substrate raw material (1);
pre-treating the culture medium, wherein the culture medium is in a solution state after pre-treatment (2);
inoculating the culture medium, wherein a mixture is obtained upon inoculation (3);
amplifying the culture in the most suitable technological condition for culture (4);
separating and extracting microbial biomass from water in the mixture (5);
collecting the water product for later use (6); and
collecting the microbial solids product (i.e., SCP) for continued processing and later use (7).

### DETAILED DESCRIPTION

A resource recovery method for controlled substrates is provided herein. The present invention relates to high-BOD by-products derived from the production of food, beverage, fuel, and other commodities. Such goods include palm oil, sugar, ethanol, biodiesel, and others. The associated by-products include concentrated syrup, distillers solubles, palm oil mill effluent, liquid honey, and glycerin. However, these by-products typically are not applicable to the direct culture of aerobic microbes. Due to high BOD, these by-products may prove unsuitable for direct treatment in a WWTF. It should be noted that BOD quantifies the amount of oxygen required to substantially convert (or oxidize) pollutants biologically in wastewater; generally speaking, BOD is associated with the amount of substrate available for metabolism and growth by microbes.

Even beyond the excessively high BOD levels exhibited by these by-products, other physical features often hinder direct input to a wastewater treatment system. For example, these by-products typically exhibit high viscosity or low water activity. These features inhibit the growth of large quantities of microbes and present difficulties with respect to operations, especially regarding maintaining sufficient concentrations of dissolved oxygen (DO) for aerobic biochemical conversion.

With such difficulties inherent to aerobic conversion processes, anaerobic metabolism does provide an option for removing BOD. However, microbial biomass yield is very low for anaerobic processes. Consequently, the yields of protein, nucleic acids, nucleotides, vitamins, and other nutrients remain low as well. For example, a typical anaerobic microbial biomass yield from BOD is about 0.2 whereas aerobic biomass yields generally surpass 0.5. Further, material transfer may be inhibited under anaerobic conditions, as can be the case with CDS, POME, and glycerin as well as for gel and peptone production. Additionally, partially due to low yields, conversion processes using anaerobic microbes tend to carry cell ages significantly longer than corresponding aerobic processes, resulting in poorer nutritional quality for any generated SCP product.

For reasons such as those above, aerobic conversion processes represent a better alternative for producing SCP. However, in the case of high-BOD by-products, aerobic processes generate significant heat that must be dissipated in order to maintain conditions appropriate for the mesophilic microbes commonly associated with biochemical conversion and wastewater treatment processes. To meet cooling requirements, a proper heat dissipation system must accompany such high-BOD systems in order to ensure that the growth of mesophilic microorganisms is maintained within an ideal temperature range between 20° C. and 45° C. Heat dissipation strategies may include exchangers, cooling towers, and other methods.

However, such cooling systems can prove costly, both from an infrastructure and operations perspective. Consequently, operating a high-BOD biochemical conversion process at elevated temperatures may prove more cost-effective. Hence, the present invention relates to the culture and use of thermophilic microbes, which grow and reproduce best within a temperature range between 41° C. and 100° C., in an industrial setting. In other words, thermophilic microbes are inoculated as the culture strain in a high-temperature environment as a strategy for making a biochemical conversion process economically feasible.

To reiterate, the present invention involves controlled substrates such as concentrated syrup and glycerin. These by-products may exhibit high BOD, high viscosity, low water activity, or low pH. Accordingly, in their typical industrial form, these controlled substrates cannot be treated biochemically according to earlier patents. Simply put, in this unaltered form, such controlled substrates prove recalcitrant to biochemical conversion. However, per the present invention, these controlled substrates may become amenable to producing food-grade SCP following any combination of dilution, pH adjustment, or nutrient addition.

### PREFERRED EMBODIMENT

Referring back to the FIGURE, the process begins with a culture medium made from controlled substrate (1). Most typically, the controlled substrate is either CDS or POME, but can also be glycerin or other high-BOD materials with poor availability to biochemical conversion in their raw form. In some cases, these controlled substrates may even exist in the form of powder or flake. Depending on particle sizing, it may prove beneficial to pulverize or mill the material in order to make the material more available to microbes. Similarly, hydrolysis may be employed for the same purpose.

Regardless, the next step involves pre-treatment of the culture medium (2). Most important, this step involves dilution of the high-BOD material or dissolving solid powdered material as may be the case. It should also be noted that the dilution water may very well derive from the product water (7) generated at the back end of the process. In the example of CDS, controlled substrate is blended with dilution water in order to achieve a final concentration between 10,000 mg/L and 90,000 mg/L, with a preferred BOD on the order of 20,000 mg/L. The actual targeted concentration depends on the volume of tankage available in the downstream process as well as the operational temperature and saturation value for DO. Optimum dilution is achieved in line with ensuring complete metabolism of BOD in the downstream amplification step (4). Also dependent on temperature is the need to provide thermal control at this step. In the case where thermophilic metabolism will be maintained downstream, it is necessary to ensure that the temperature of the culture medium remains compatible (i.e., will not cool the amplification process to below the thermophilic range). In the case where mesophilic metabolism will be maintained downstream, it is necessary to ensure that the temperature of the culture medium remains compatible (i.e., will not heat the amplification process above the mesophilic range).

The subsequent step entails inoculation (3) of the culture medium with the desired microbial culture. This culture may well be developed in a small fermentation tank serving to “seed” the larger process. Alternatively, this culture may be recycled or returned from later stages of the process such as at separation (5). In the case of using a small fermentation tank, mixed bacteria are cultivated for 3 to 8 hours in a low-concentration culture solution. During this period, the strain secretes extracellular polymeric substances to absorb nutrients from the culture solution. As this substance accumulates, the community forms a visible flocculent cluster. At this point, the culture development is complete and the bacteria are ready for seeding the larger process. The typical inoculation ratio falls between 10 and 100 litres of inoculum per cubic meter of wastewater.

When the culture medium is inoculated with a microbial culture, the inoculated microbes include but are not limited to at least two of the species sphingobacteria, comamonas, xanthomonas, microbacterium, flavobacterium, alcaligenes, porphyromonas, saprospira, and Rhodopseudomonas palustris. The inoculated microbes are subdivided from family to genus, including but not limited to at least one of Lewinella, Parapedobacter, Emticicia, Luteibacter, Thermomonas, Denitrobacter, Comamonas, Chiyseobacterium, Microbacterium, Dysgonomonas, Acinetobacter, and Curvibacter. The inoculated microbes are subdivided from genus to species, including but not limited to at least one of Lewinella marina, Parapedobacter koreensis, Emticicial oligotroghica, Luteibacter anthropi, Curvibacter gracilis, Dysgonomonas wimpennyi and Thermomonas koreensis.

Next, the inoculated culture medium passes to the amplification stage (4). In classical microbiological terms, this is where fermentation or growth take place. And this is the step where operational parameters must be maintained, specifically DO, temperature, and pH. Particularly in the case of POME, it is important to avoid depressed pH; such conditions are avoided most typically by adding sodium hydroxide or some other hydroxyl-containing compound. When the culture is amplified, the mixture is supplied with nutrients and micronutrients and is continuously mixed. Nutrient sources include urea and monopotassium phosphate, which are added such that the ratio of organic carbon to total nitrogen to total phosphorus=100:10:1. Micronutrients include elements such as magnesium, zinc, manganese, boron, and others. As indicated earlier, this step may be temperature-controlled (e.g., between 10° C. and 40° C. for mesophiles), is pH-controlled (e.g., between potenz values of 5.5 and 8.5), and is provided with air and oxygen (e.g., at a rate of 0.02 to 0.2 m³ of air per cubic meter of mixture per hour) for aerobic fermentation until a fermentation end-point is achieved at a redox potential (determined using an installed redox potentiometer) between +260 and +300 mV. At this point, effectively all BOD has been removed from solution and nutrients in the mixture are essentially exhausted. As was the case with the inoculum, extracellular polymeric substances secreted from the strain ensure the formation of the stable flocculent bacteria clusters.

The next unit operation is separation (5) of the two products (i.e., treated water and microbial solids). With BOD exhausted from the liquid phase, the solids now contain much of the organic carbon originally found in the controlled substrate. When the culture is separated, mixing and oxygenation are stopped, and the mixture is divided into a liquid supernatant water product and flocculent clusters of microbial solids using methods such as at least one of precipitation (e.g., for a period of 0.5 to 4 hours), filtration, concentration, centrifugation, or other processes.

When the water product (6) is separated, it may be processed further using techniques such as ultrafiltration, nanofiltration, reverse osmosis, ion exchange, or other processes in order to render it appropriate for subsequent use for washing, cooling, dilution, make-up, irrigation, agriculture, aquaculture, or other purposes.

When the microbial solids (7) are separated into their own discrete SCP product, they may be dewatered and processed further using techniques such as cell lysis, enzymatic hydrolysis, drying, sterilization, or other processes in order to create a product for subsequent use as feed, food, fertilizer, or soil amendment. The final SCP product of concentrated flocculent microbial clusters may be used as an aquatic animal protein feed following subsequent processing including cell lysis, enzymatic hydrolysis, drying, sterilization, and others.

As a result of producing these two products, both liquid and solid, the process is largely free of generating further waste.

### Additional Embodiments

### Regarding the Culture Medium (1):

In some embodiments, the controlled substrates are the by-products derived from the production of food, beverage, and other forms of biological conversion. Further, in some embodiments, the controlled substrates are from: a) by-products of bio-fuel production; b) by-products of medicine production; c) by-products of fertilizer production; or d) by-products of chemical production.

In some embodiments, the by-products of food, beverage and/or bio-fuel maintain their food-grade qualities. This is accomplished through processing procedures including but not limited to a) avoiding mixing of by-products with other materials, including other by-products or waste; b) conveying these separated by-products via devoted means and then storing them in their own devoted tanks, etc.; c) utilizing food-grade pumps, pipelines, and other delivery equipment; d) storing these by-products in containers with appropriate linings, seals, and covers to avoid contamination from the air or other environmental sources; e) ensuring only food-grade microbes are used for inoculation; f) using only food-grade nutrients for subsequent additions in the process.

In some embodiments, a small reactor is used for culturing the microbial inoculum. The culture substrate of the microbial inoculum includes the controlled substrates used during the culture amplification. In some embodiments, the microbial inoculum culture is controlled so as only to produce a specific, known community of microbes. The species characteristics of this community are maintained by avoiding contamination through additions including air, water, and solids. The quality of the community is monitored through observation of morphological characteristics and plate-count methods. In some embodiments, the first bacteria introduced into the reactor grow rapidly at an early period of inoculation. However, over time this “pioneer” inoculum may cease growing or even wash out of the reactor. This condition is still considered relevant to culture development for the present invention.

### Regarding Pre-Treatment of Culture Medium (2)

In some embodiments, the by-products of food, beverage and/or bio-fuel are diluted to be applicable to the culture of the microbes. In some embodiments, water may be used as a solute or for dilution that contains other materials applicable to the growth of microbes. In addition to liquid ingredients, the culture medium can further include salt, microelements, protein, lipids, and can further include exogenous soluble organic material.

The controlled substrates are added into the medium in a liquid or solid form. In some embodiments, controlled substrate powder or flake is added into the liquid medium, and then fully stirred to obtain the mixed liquid culture substrate. In such cases where controlled substrates are added in a dried form, oxygen and/or air is fed into the medium, and then mixed and diffused via bubbles. In such cases, the temperature may be set at 25° C. or higher to reduce viscosity below 30 CP, enabling better mixing of controlled substrates and the liquid medium.

In some embodiments, controlled substrates are dissolved into the liquid medium or the controlled substrate already contain large volumes of that liquid medium without the need to add further dilution water. In the case of blending such liquids, a BOD concentration between 15,000 mg/L and 25,000 mg/L is favorable.

In some embodiments, when the BOD concentration of the culture substrate is very high, for instance the BOD concentration is higher than 100,000 mg/L or even 1,000,000 mg/L, favorable dilution results in a final BOD concentration range of 10,000-40,000 mg/L. In some embodiments, the BOD concentration is adjusted to be about 10,000 mg/L, 20,000 mg/L, 30,000 mg/L, 40,000 mg/L, 50,000 mg/L, 60,000 mg/L, 70,000 mg/L, 80,000 mg/L, 9,000 mg/L, and any determined value between 10,000 mg/L and 90,000 mg/L.

In some embodiments, the by-products of food, beverage and/or bio-fuel are enzymatically hydrolyzed using an exogenous enzyme such as amylase, cellulase, lipase, hemicellulose, glucanase, or other similar enzyme. The microbial suitability of the by-products of food, beverage and/or bio-fuel and/or the culture substrate pre-treatment are improved by such enzymatic hydrolysis. Additionally, hydrolysis and/or “steam fermentation” technologies may be applied. In some embodiments, the glucanase can be applied to lower the content of glucan in the by-products of food, beverage and/or bio-fuel rich in glucan, thereby improving by-product pretreatment conditions.

In some embodiments, the by-products of food, beverage and/or bio-fuel will be pulverized to shorten by-product particles. The pulverization method may comprise use of a colloid mill, cone mill, wet mill, or other related pieces of equipment. In some embodiments, at least 50% of the crushed by-products may be converted into colloidal substances, with particles shortened to 0.45 μm or less. Such smaller particles may be metabolized more rapidly by bacteria within a 24-hour period. Such pulverization or crushing can be applied before or after enzymatic hydrolysis and may even take the place of enzymatic hydrolysis.

What is claimed is:

1. A resource recovery method, comprising:
a culture medium with controlled substrates;
pre-treating the culture medium, wherein the culture medium is in a solution state after pre-treatment;
inoculating the culture medium, wherein a mixture is obtained upon inoculation;
amplifying the culture in the most suitable technological condition for culture;
separating and extracting microbial biomass from the mixture,
wherein
when the culture medium is inoculated, the inoculated microbes include but not only limited to at least two of sphingobacteria, comamonas, xanthomonas, microbacterium, flavobacterium, alcaligenes, porphyromonas, saprospira and Rhodopseudomonas palustris;
when the culture is amplified, the mixture is stirred continuously and is filled with compressed air/oxygen for aerobic fermentation, and a redox potential +260 to +300 is taken as a fermentation end-point;
after the aerobic fermentation, stirring and filling of the compressed air are stopped, the mixture is separated into a liquid supernatant and a flocculent bacteria cluster.

2. The resource recovery method according to claim 1, wherein the flocculent bacteria cluster is taken as a low-concentration culture solution of a mixed bacteria inoculum.

3. The resource recovery method according to claim 1, wherein the concentrated flocculent bacteria cluster can be produced as aquatic animal protein feed and food raw materials upon subsequent cell lysis, enzymatic hydrolysis, drying and sterilization.

4. The resource recovery method according to claim 1, wherein the above liquid supernatant is used for, but not only limited to, at least one of wash water for production processes, water for irrigation, water for fish farming, or water to replenish natural resources.

5. The resource recovery method according to claim 1, wherein the liquid supernatant sterilized and softened is used for, but not limited to, at least one of water for landscape environment, municipal water, production cooling water, boiler water, or process water.

6. The resource recovery method according to claim 1, wherein between 0.02 and 0.2 m³ compressed air is filled into each cubic meter of mixture per hour.

7. The resource recovery method according to claim 1, wherein a fermentation temperature is maintained between 0 and 40° C. and a pH value of the mixture is maintained between 5.5 and 8.5 during aerobic fermentation.

8. The resource recovery method according to claim 1, wherein a redox potentiometer is installed to measure the redox potential.

9. The resource recovery method according to claim 1, wherein the method of separating the mixture into the liquid supernatant and the flocculent bacteria cluster includes but is not limited to at least one of precipitation, filtration, concentration, or centrifugation.

10. The resource recovery method according to claim 1, wherein after the aerobic fermentation, the mixture is statically precipitated for a period between 0.2 and 4 h so that the flocculent bacteria cluster in the mixture is precipitated and formed into bacteria solids, and then the mixture is separated to obtain the liquid supernatant and the flocculent bacteria cluster respectively.

11. The resource recovery method according to claim 1, wherein the controlled substrate derives from one the following industries: food production, feed production, biofuel production, medicine production, or chemical production.

12. The resource recovery method according to claim 1, wherein the controlled substrate may not be amenable to microbial conversion due to properties including: elevated BOD, elevated viscosity, or low pH.

13. The resource recovery method according to claim 1, wherein pre-treatment of controlled substrate to render it amenable to microbial conversion includes one of the following strategies: dilution, pulverization, hydrolysis, or temperature increase.

14. The resource recovery method according to claim 1, wherein thermophilic microbes provide the means of biologically converting water-borne compounds into SCP.

15. The resource recovery method according to claim 1, wherein the concentrated flocculent bacteria cluster can be produced as fertilizer or a soil amendment.
